SEQUENCE LISTING (6 sequences)

SEQ ID NO: 1 - DNA, 26 bp, Artificial Sequence
  Description of Artificial Sequence Hind III site and Kozak sequence-containing primer designed for the amplification of cyclophilin A DNA
gcaagcttaccatggtcaaccccacc

SEQ ID NO: 2 - DNA, 26 bp, Artificial Sequence
  Description of Artificial Sequence Bam HI site-containing primer designed for the amplification of cyclophilin A DNA
gcggatccgagttgtccacagtcgga

SEQ ID NO: 3 - DNA, 21 bp, Artificial Sequence
  Description of Artificial Sequence mutagenic forward primer for substituting arginine with alanine at position 55 of cyclophilin A
tcctttcacgcgattattcca

SEQ ID NO: 4 - DNA, 21 bp, Artificial Sequence
  Description of Artificial Sequence mutagenic reverse primer for substituting arginine with alanine at position 55 of cyclophilin A
tggaataatcgcgtgaaagga

SEQ ID NO: 5 - DNA, 21 bp, Artificial Sequence
  Description of Artificial Sequence mutagenic forward primer for substituting tryptophan with phenylalanine at position 121 of cyclophilin A
aagactgagttcctggatggc

SEQ ID NO: 6 - DNA, 21 bp, Artificial Sequence
  Description of Artificial Sequence mutagenic reverse primer for substituting tryptophan with phenylalanine at position 121 of cyclophilin A
gccatccaggaactcagtctt

(19) United States
(12) Patent Application Publication
(10) Pub. No.: US 2005/0074438 A1
(43) Pub. Date: Apr. 7, 2005

(54) Use of cyclophilin as antioxidant and prevention of cyclosporin a-induced toxicity in cell transplantation by overexpression of cyclophilin

(76) Inventors: Sung Soo Kim, Seoul (KR); Jin Hwa Lee, Busan (KR); Feng Hong, Seoul (KR); Joo Hun Ha, Seoul (KR)

Correspondence Address: MUSERLIAN, LUCAS AND MERCANTI, LLP, 475 PARK AVENUE SOUTH, 15TH FLOOR, NEW YORK, NY, 10016, US

(21) Appl. No.: 10/635,264

(22) Filed: Aug. 6, 2003

(30) Foreign Application Priority Data
Nov. 11, 2002 (KR) 10-2002-0069822

(51) Int. Cl. A61K 48/00; C12N 5/08

(52) U.S. Cl. 424/93.21; 435/366

(57) ABSTRACT

Disclosed is a cyclophilin protein with PPIase activity functioning as an antioxidant. When being overexpressed in transplanted cells, the cyclophilin protein remarkably reduces the cytotoxicity induced by cyclosporin A or its analogues so that it can greatly improve the success rate in transplantation. Also, disclosed is a composition useful to prevent transplant rejection, comprising a recombinant expression vector which can over-express a cyclophilin protein with PPIase activity. The recombinant expression vector is introduced into cells which are thus transformed to be resistant to cyclosporin A and its analogues. A method of preparing such cells is also disclosed.

### BACKGROUND OF THE INVENTION

1. Field of the Invention

The present invention relates to the use of cyclophilin with peptidyl-propyl-cis-trans isomerase (PPIase) activity as an antioxidant and a method of preventing immunosuppressant cyclosporin A (CsA)-induced toxicity in cell transplantation by overexpressing cyclophilin.

2. Brief Description of the Prior Art

CsA (Cyclosporin A) is a potent immunosuppressant that is widely used in organ transplantation and autoimmune diseases (Alejandro, D. S., et al., J. Am. Soc. Nephrol. 5, 153-160, 1994). CsA is a cyclic undecapeptide that bind to cyclophilin A (CypA) with a high affinity. CypA is a cytosolic protein with PPIase (peptidyl-propyl-cis-trans isomerase) activity that is potently inhibited by CsA binding. PPIase enzymes function as molecular chaperones to faciliate protein folding, intracellular trafficking and maintenance mult-protein complex stability (Andreeva, L., et al., Int. J. Exp. Pathol. 80, 305-315, 1999; Hamilton, G. S., et al., J. Med. Chem. 41, 5119-5143, 1998), although it is believed that inhibition of PPIase activity is not required for its immunosuppressive action (Bierer, B. E., et al., Science 250, 556-559, 1990). CsA-CypA complex, but not CypA alone, binds and inhibits the activity of calcineurin, which is a calcium/calmodulin-dependent protein phosphates. (Friedman, J., et al., Cell 66, 799-806, 1991; Liu, J., et al., Biochemistry 31, 3896-3901, 1992). The inhibitor of calcineurin activity blocks the translocation of NFATs (nuclear factors of activated T-cells), which in turn prevents T-helper cells from expressing several lymphokines that mediate the activation of immune reaction (Matsuda, S., et al., Immunopharmacology 47, 119-125, 2000).

Although CsA made an exceptional turning point in transplantation and autoimmune disease treatment, its toxicity in various tissues including skeletal muscle caused the patients serious troubles (Rush, D. N., Clin. Biochem. 24, 101-105, 1990; Arellano, F., et al., Lancet 337, 915, 1991; Biring, M. S., et al., J. Appl. Physiol. 84, 1967-1975, 1998). CsA-induced myopathies clinically cause myalgia, cramps, muscle weakness and elevation of plasma creatine kinase. (Goy, J. J., et al., Lancet 1, 1446-1447, 1989; Fernadex-Sola, J., et al., Lancet 335, 362-363, 1990). There have been many attempts to reveal the mechanism of CsA-induced myopathy. Several groups have proposed that CsA hinders muscle regeneration from satellite myoblasts since CsA was shown to induce muscle deficiency in regenerated muscle fibers, and to inhibit myogenic differentiation in cultured myoblasts (Hardiman, O., et al., Neurology 43, 1432-1434, 1993; Abbott, K. I., et al., Mol. Biol. Cell 9, 2905-2916, 1998; Friday, B. B., et al., J. Cell Biol. 149, 657-666, 2000). Other studies revealed the activities of CsA to decreases mitochondrial respiration (Hokanson, J. F., et al., Am. J. Respir. Crit. Care. Med. 151, 1848-1851, 1995) and to inhibit creatine uptake as a result of creatine transporter reduction (Tran, T. T., et al., J. Biol. Chem. 275, 35708-35714, 2000), suggesting that CsA-induced myopathies might be related to mitochondrial injury or altered energy states.

Myoblasts or satellite cells represent a population of myogenic stem cells that can proliferate and fuse to or replace damaged muscle myofibers. Using dy/dy and mdx mouse models of genetic myopathies, it was shown in the late 1980's that normal genes could be introduced into dystrophic muscles of genetic myopatheis (Law, P. K. et al., Muscle Nerve 11, 525-533, 1998; Partridge, T. A. et al., Nature 337, 176-179, 1989). On the basis of this finding, allogenic myoblast transplantation (AMT) was tried in patients suffering from Duchenne dystrophy, typical of severe hereditary muscle disorders. Unfortunately, the initial clinical trials were unsuccessful (Skuk, D. et al., Microsc. Res. Tech. 48, 213-222, 2000). Since then, many research groups took up the study of improving the survival rate of transplanted myoblasts using animal models. One of the results obtained is that transplanted myoblasts are primarily rejected by immune response (Gill, R. G. et al., Cell Transplant 4, 361-370, 1995). Thus, it was found that the success of AMT needs a suitable immunosuppressant to be administered prior to transplanting myoblasts.

Known as immunosuppressants useful for the transplantation of myoblasts are CsA, tacrolimus (FK506) and cyclophosphamide. FK506 showed a success rate of 95% or higher both in clinical trials and animal model studies (Kinoshita, I. et al., Muscle Nerve 17, 1407-1415, 1994; Kinoshita, I. et al., Muscle Nerve 18, 1217-1218, 1995), while cyclophosphamide did not increase the survival of the fused muscle fibers after AMT (Karpati, G. et al., Ann. Neurol. 34, 8-17, 1993; Vilquin, J. T. et al., Neuromuscul. Disord. 5, 511-517, 1995), and CsA ensuresd only a moderate success rate for the transplantation (Labrecque, C. et al., Transplant. Proc. 24, 2889-92, 1992).

Although CsA is a potent immunosuppressant marking a turning point in transplantation, it has been used with great reluctance due to its low success rate associated with the following problems. CsA causes cytotoxicity in various types of cells through reactive oxygen species (ROS) (Wang, C. et al., Transplantation 58, 940-946, 1994; Perez de Lema, G. et al., Life Sci. 62, 1745-1753, 1998; Wolf, A. et al., J. Pharmacol. Exp. Ther. 280, 1328-1334, 1997). Further, CsA is found to give rise to muscle depletion in regenerated muscle fibers and inhibit muscle differentiation in myoblast cultures, thereby blocking muscle regeneration from satellite myoblasts (Hardiman, O. et al., Neurology 43, 1432-1434, 1993; Abbott, K. L. et al., Mol. Biol. Cell, 9, 2905-2916, 1998; Friday, B. B. et al., J. Cell Biol., 149, 657-666, 2000).

With the aim of raising transplantation success rates, attempts have been made to remove CsA-induced toxicity. For instance, antioxidants were used together with CsA (Wang, C. et al., Transplantation 58, 940-946, 1994; Perez de Lema, G. et al., Life Sci. 62, 1745-1753, 1998; Kumar, K. V. et al., Transplantation 67, 1065-1068, 1999; Naidu, M. U. et al., Nephron. 81, 60-66, 1999). Antioxidants could partially protect cells from the apoptosis attributed to intracellular ROS increase, but could not prevent the blockage of cell differentiation.

Accordingly, there remains a need for better alleviating the cellular toxicity induced by CsA so as to improve the success rate of cell transplantation.

### DISCLOSURE OF THE INVENTION

The thorough and intensive research into the alleviation of CsA-induced toxicity, conducted by the present inventors, resulted in the finding that the CsA-induced toxic effect on transplanted cells, that is, the apoptosis and cell differentiation blockage of transplanted cells, is caused by oxidative stress, at least partly via inhibition of the PPIase activity of CypA. Indicating that the PPIase activity of CypA is directly involved in cell differentiation, this finding is in contrast to the conventional assertion that the CsA-induced cell differentiation blockage results from the inhibition of calcineurin activity. Also, the present inventors found that the cells that have survived after pre-exposure to CsA could not only reversibly proliferate and differentiate, but also are resistant to subsequent CsA exposure. On the basis of these findings, the present inventors established that the CsA-induced cytotoxicity effect on transplanted cells can be remarkably reduced by the overexpression of CypA in the cells to be transplanted.

In accordance with an aspect of the present invention, there is provided an antioxidant, comprising a cyclophilin protein with PPIase activity.

In accordance with another aspect of the present invention, there is provided a pharmaceutical composition for preventing cyclosporin A-induced cytotoxicity by the overexpression of cylcophilin with PPIase activity in the transplanted cells, comprising a recombinant expression vector which can express the cyclophilin protein in such a sufficient amount as to reduce the toxicity induced by cyclosporin A or its analogues.

In accordance with a further aspect of the present invention, there is provided a cell for use in the transplantation which is resistant to cyclosporin A or its analogues, wherein a cyclophilin protein with PPIase activity is over-expressed.

In accordance with still another aspect of the present invention, there is provided a method of preparing the cells for use in the transplantation which is resistant to cyclosporin A or its analogues, comprising the steps of introducing a gene encoding a cyclophilin protein with PPIase activity into a vector to construct a recombinant expression vector, transfecting the recombinant expression vector into the cells for transplantation, culturing the transfected cells, and selecting cells in which the cyclophilin with PPIase activity is over-expressed.

In accordance with still a further aspect of the present invention, there is provided a method of preparing the cells for use in the transplantation which are resistant to cyclosporin A or its analogues, comprising the steps of culturing the cells for transplantation in the presence of cyclosporin A or its analogues and recovering viable cells from the cultures.

### BRIEF DESCRIPTION OF THE DRAWINGS

FIG. 1 is a histogram showing the effects of CsA, FK506 and asciomycin on calcineurin phoaphatase activity.

FIG. 2a provides microphotographs showing morphological changes of myoblasts treated with CsA, SDZ NIM811, FK506 and ascomycin to examine the effects of the drugs on the differentiation of rat cardiac myoblasts.

FIG. 2b provides results of the Western blotting analysis in which rat cardiac myoblasts is treated with CsA, SDZ NIM811, FK506 and ascomycin to examine the effects of the drugs on the differentiation of myoblasts.

FIG. 2c provides fluorescent microphotographs of the TUNEL analysis in which rat cardiac myoblasts are treated with no drug (a, control), 1 μM CsA (b), 2.5 μM CsA (c), 5 μM CsA (d), 10 μM CsA (e), 1 μM SDZ NIM811 (f), 1 μM SDZ MIM811 (g), 9 μM FK506 (h) and 200 nM ascomycin (i) to examine the effects of the drugs on the cell death of myoblasts.

FIG. 2d is a photograph taken after a DNA fragmentation analysis is performed using rat cardiac myoblasts treated with CsA, SDZ NIM811, FK506 and ascomycin to examine the effects of the drugs on the cell death of myoblasts.

FIG. 3a provides microphotographs showing morphological changes of the myoblasts originated from rat limb skeletal muscles when the cells are treated with CsA, SDZ NIM811, FK506 and ascomycin to examine the effects of the drugs on the differentiation of myoblasts.

FIG. 3b provides results of the Western blotting analysis in which rat limb skeletal muscle-derived myoblasts is treated with CsA, SDZ NIM811, FK506 and ascomycin to examine the effects of the drugs on the differentiation of myoblasts.

FIG. 3c provides fluorescent microphotographs of the TUNEL analysis in which rat limb skeletal muscle-derived myoblasts are treated with no drug (a, control), 1 μM CsA (b), 2.5 μM CsA (c), 5 μM CsA (d), 10 μM CsA (e), 1 μM SDZ NIM811 (f), 1 μM SDZ MIM811 (g), 9 μM FK506 (h) and 200 nM ascomycin (i) to examine the effects of the drugs on the cell death of myoblasts.

FIG. 3d is a photograph taken after a DNA fragmentation analysis is performed using rat limb skeletal muscle-derived myoblasts treated with CsA, SDZ NIM811, FK506 and ascomycin to examine the effects of the drugs on the cell death of myoblasts.

FIG. 4 provides microphotographs showing morphological changes of CsA-treated cells during muscle differentiation to examine the effect of CsA on muscle differentiation and cell death.

FIG. 5a provides FACS plots in which ROS levels in myoblasts cultured in PM (a), in DM (b), in DM treated with CsA (c), in DM treated with SDZ NIM811 (d), in DM co-treated with CsA and DFOM (deferoxamine mesylate) (e), and in DM co-treated with CsA and catalase (f) are measured for ROS levels to examine if ROS is involved in the toxicity induced by CsA during muscle differentiation.

FIG. 5b provides microphotographs showing morphological changes of cells treated with 10 μM CsA (control), with both 0.4 mM DFOM and 10 μM CsA (DFOM) and with both 2000 units/ml catalase and 10 μM CsA (catalase) and a histogram showing % cell survival of the cells.

FIG. 6a provides results of the Western blotting analysis using cells treated with various drugs, and a histogram in which the Western blots are quantified to examine the effect of CsA on the expression of Bax.

FIG. 6b provides FACS plots in which cells cultured in PM (a), in DM (b), and in DM containing 9 μM FK506 (c), 200 nM ascomycin (d), 1 μM CsA (e), 2.5 μM CsA (f), 5 μM CsA (g) and 10 μM CsA (h) are measured for mitochondria membrane potential.

FIG. 7a provides results of the Western blotting analysis using gene-transfected cells, and a histogram in which the Western blots are quantified to examine CypA expression levels.

FIG. 7b is a histogram in which gene-transfected cells are measured for doubling time.

FIG. 7c provides FACS plots in which gene-transfected cells cultured in the presence or absence of CsA are measured for ROS content.

FIG. 7d provides microphotographs showing morphological changes of gene-transfected cells after being exposed to CsA and H₂O₂.

FIG. 7e is a histogram showing % cell survival of gene-transfected cells after being exposed to CsA and H₂O₂.

FIG. 7f provides Western blot analysis results for Bax expression levels after gene-transfected cells are exposed to CsA and H₂O₂.

FIG. 8a provides microphotographs showing morphological changes of gene-transfected cells upon completion of muscle differentiation.

FIG. 8b provides microphotographs showing the effect of antioxidants on muscle differentiation.

FIG. 9a provides results of the Western blot analysis in which cells are treated with CsA to examine if CsA induces CypA expression.

FIG. 9b provides microphotographs showing morphological changes of cells pre-exposed to CsA to examine if the pre-exposed cells can proliferate and differentiate reversibly when being again exposed to CsA.

### DETAILED DESCRIPTION OF THE INVENTION

CsA is known to bind to mitochondrial protein, cyclophilin D (CypD) to block the MPTP (mitochondrial membrane permeability transition pore) and, thereby, inhibit apoptosis (Szabo., I. et al., J. Biol. Chem., 266, 3376-3379, 1991; Halestrap, A. P. et al., Biochem. J. 268, 153-160). In contrast, some studies demonstrate that CsA induces oxidative stress (Longoni, B., et al., FASEB J. 15, 731-740, 2001; Leonardi, A., et al., Arch. Ophthalmol. 119, 1512-1517, 2001; Zhong, Z., et al., Am. J. Physiol. 275, F595-604; Zhong, Z., et al., Mol. Pharmacol, 56, 455-463). Also, some studies suggest that because CsA-induced cytotoxicity is reduced by antioxidants, reactive oxygen species (ROS) mighy be important factors of CsA-induced cytotoxicity (Wang, C., et al., Transplantation 58, 940-946, 1994; Kumar, K. V., et al., Transplantation 67, 1065-1068, 1999; Naidu, M. U., et al., Nephron, 81, 60-66, 1999).

Consistent with such research results, data obtained through the experiments conducted by the present inventors demonstrate that CsA causes differentiation blockage and apoptosis in the early stage of muscle differentiation through the generation of ROS. The extent of stress was found to be proportional to the concentration of CsA, as measured by ROS level. Cells underwent apoptosis proportionally in the CsA concentration range from 1 to 10 μM, and muscle differentiation was completely blocked at 2.5 μM of CsA. Treatment with antioxidants prevented apoptosis, but could not impede the process into differentiation blockage. According to CsA treatment, Bax expression was increased while mitochondrial membrane potential was decreased. These results indicate that CsA-induced apoptosis might be related to mitochontrial lesion. Such CsA effects, which apparently contradict each other, can be explained by the CsA actions which vary with concentration. At nanomole levels of ROS, which is almost free of toxicity, CsA is reported to show anti-apoptosis activity. By contrast, in the ROS concentration of 1 μM or higher, CsA causes oxidative stresses to cells which then open MTPT in an unregulated manner which does not respond to CsA (He, L., et al., FEBS Lett. 512, 1-7). Little is known about a detailed mechanism in which CsA alters Bax expression, mitochondrial membrane potential and caspase-3 expression and causes apoptosis.

In spite of the knowledge that ROS mediates CsA-induced cytotoxicity, the molecular level mechanism of the ROS generation is not clearly delineated. CypA, an intracellular target of CsA, is suggested to have an anti-ROS function (Doyle, V., et al., Biochem. J. 341, 127-132, 1999; Lee, J. P., et al., Proc. Natl. Acad. Sci., U.S.A., 96, 3251-3256). According to the experiment data of the present inventors, CsA-mediated ROS generation is decreased in CypA/WT cells (in which a CypA gene of the wild type rat brain is transfected into H9c2 rat cardiac myoblast) and CypA/R55A cells (in which a mutated CypA gene of the wild type rat brain is transfected into H9c2 rat cardiac myoblast, the mutated CypA gene expressing a CypA protein which has an alanine residue instead of an arginine residue at position 55 and shows one thousandth PPIase activity compared to the wild type protein) undergo more severe oxidative stresses than mock transfected cells. These data imply that PPIase activity is closely related to the anti-ROS function of CypA and consequently, CsA generates ROS by at least partially inhibiting PPIase activity. The CypA's anti-oxidative function attributed to the SH group sensitive to reduction/oxidation reaction (Lee, S. P., et al., J. Biol. Chem. 276, 29826-29832) may need the PPIase activity because CyopA/R55A keeps the SH group intact although losing its ROS scavenging function.

According to the present invention, the PPIase activity is understood to play a direct role in muscle differentiation, based on the finding that the overexpression of dominant negative CypA/R55A completely blocks muscle differentiation independently of CsA treatment. Practically, it may be estimated that CypA/R55A generates ROS to such a toxic level as to block muscle differentiation, since differentiating myoblasts generate a significant amount of ROS and the overexpression of CypA/R55A increases the ROS level in a differentiation medium (DM). However, the present invention reveals the direct involvement of the PPIase activity independent of anti-oxidative activity in muscle differentiation on the basis of the following facts. To begin with, the ROS level of CypA/R55A cells in a DM is comparable to that of mock transfected myoblast treated with 1 mM CsA in a DM where muscle differentiation is partially blocked. Also, CypA/WT, CypA/W121F (in which a mutated CypA gene of the wild type rat brain is transfected into H9c2 rat cardiac myoblast, the mutated CypA gene expressing a CypA protein which has a phenylalanine residue instead of a tryptophan residue at position 121 and shows one hundredth affinity for CsA and half PPIase activity compared to the wild type protein) and antioxidants decrease ROS levels to protect cells from CsA-induced apoptosis, but cannot reverse the differentiation block. In a DM, muscle differentiation requires physiological ROS generation. In this case, the reduction of ROS below the level necessary for muscle differentiation by use of CypA/WT, CypA/W121F or antioxidants could be the single most important reason. However, although the ROS level was titrated to a proper extent by using antioxidants in various concentrations, CsA-caused differentiation blockage did not disappear. In contrast, the differentiation blockage induced by other oxidants such as menadione and doxorubicin could be prevented by controlling the ROS level. The fact that the differentiation blocking effect of CsA-induced cytotoxicity cannot be prevented even by lowering the ROS level to the physiological concentration implies that the CypA-PPIase activity is inhibited by CsA. Since the CypA-PPIase activity functions as a molecular chaperone (Andreva, L., et al., Int. J. Exp. Pathol. 80, 305-315), it may be assumed that CsA might block the muscle differentiation by interrupting a proper folding of myogenic proteins. Such direct and indirect requirement for CypA-PPIase activity shows that the blockage of CypA-PPIase activity is a major mechanism of CsA-mediated differentiation blockage. These findings of the present inventors apparently contradict to the previous reports suggesting that CsA blocks muscle differentiation by inhibiting the activity of calcineurin (Friday, B. B., et al., J. Cell Biol., 149, 657-666). In the present invention, it is revealed that SDZ NIM811, a CsA analogue lacking calcineurin inhibitory activity, exhibits the same cytotoxicity as that of CsA and that FK506 and ascomycin do not cause differentiation blockage even at a concentration sufficient to completely inhibit the transiently increased calcineurin activity during muscle differentiation. These data demonstrate that the transiently increased calcineurin activity is not indispensable for muscle differentiation.

Recently, oxidative stress-caused CypA induction has been reported (Hovland, A. R., et al., Neurochem. Int. 35, 229-235). According to experimental data obtained by the present inventors, CsA-caused CypA induction peaks at a CypA concentration of 5 μM with the occurrence of complete differentiation blockage and the death of half the cells. Myoblasts pre-exposed to CsA reversibly proliferate and differentiate in a proliferation medium (PM) and a DM, respectively, each containing a higher CsA concentration, as well as showing resistance upon re-exposure to CsA. These results indicate that induction of CypA expression plays an important role in the adaptation to CsA. The adaptation in which CypA is induced in the presence of a low concentration of CsA enables myoblasts to differentiate following subsequent exposure to a higher CsA concentration. In contrast, CypA/wt and CypA/W121F cells are protected from apoptotic death in the presence of 10 μM CsA, but fail to differentiate. Naturally, the adaptation to CsA is thought to be important in vivo. The level of CsA in blood is in the range of 0.1 to 0.6 μM in a normal state, but may transiently reach 1 μM or higher (such a concentration as to trigger apoptotic cell death) during the fluctuation of blood level due to a higher initial dosage immediately after administration or transplantation (Shaw, L. M., et al., Clin. Chem., 36, 1841-1846, 1990; Tegzess, A. M., et al., Transplant. Proc. 20 (Suppl. 3), 530-533, 1988). In this case, transplanted or satellite myoblasts could not survive or differentiate without adaptation. Of course, although there is some difference in the in vivo sensitivity of myoblasts to the blood level of CsA, the adaptive response may improve the survival rate of the myoblasts transplanted in AMT patients.

The present invention is based on the findings that CsA-induced cytotoxicity is caused by oxidative stress, at least partly via inhibition of CypA PPIase activity, the PPIase activity of CypA provides an ROS scavenging effect, the overexpression of CypA protects cells from CsA-induced cytotoxicity, and the PPIase activity of CypA is directly involved in muscle differentiation.

In accordance with an aspect, the present invention encompasses the use of cyclophilin proteins as antioxidants.

The term “cyclophilin proteins” as used herein is defined as naturally occurring or recombinant cyclophilin proteins or their isoforms or mutants. CypA, a receptor for CsA, was first revealed to show enzymatic activity by Fischer et al. (Fischer, et al., Biomed. Biochim. Acta, 43:1101-1111, 1984). They identified an enzyme capable of catalyzing reciprocal cis-trans conversion of proline-containing peptides from pig liver and termed it peptidyl-prolyl cis-trans isomerase (PPIase). Amino acid sequence analysis of the N-terminal peptide revealed the identity between cyclophilin and the enzyme (Lang, et al., Nature, 329:268-270, 1987). CypA is an 18 kDa cytoplasmic protein (Haendler, et al., EMBO. J., 6:947-950, 1986) which is abundantly expressed in tissues of all mammals including humans, oxen, pig, etc. (Koletsky, et al., J. Immunol., 137:1054-1059, 1986). Since the discovery of CypA, many different cyclophilins have been identified, all of which are found to have PPIase activity. Amino acid sequences of 27 or more cyclophilins are known. Cyclophilins are exemplified by cyclophilin isoforms, each comprising a highly conserved 18-kDa domain adjacent to a characteristic domain binding to microsome or membrane targets (Gething, et al., Nature, 355:33-45, 1992; Price, et al., PNAS, 88:1903-1907, 1991; Spik, et al., J. Biol. Chem. 266:10735-10738, 1991; Friedman, et al., Cell 66:23204-23214, 1991; Bergsma, et al., J. Biol. Chem. 266:23024-23214, 1991). These proteins comprise human-derived Cyp-40 (40 kDa) (Kieffer, et al., J. Biol. Chem, 266:5503-5507, 1992) and Cyp-60 (60 kDa) (Wang, et al., Biochem. J. 314:313-319, 1996) and surface-bound natural killer (NK) cell cyclophilins (150 kDa) (Anderson, et al., PNAS, UAS 90:542-546, 1993). Other examples of cyclophilins include the proteins found in many parasites such as Schistosoma mansoni (supra Koletsky, et al., J. Immunol; Klinkert, et al., Mol. Biochem. Parasitol., 75:99-111, 1995; Kiang, et al., Mol. Biochem. Parasitol., 76:73-82, 1995), Toxoplasma gondii (High, et al., J. Biol. Chem., 269:9105-9112, 1994) and Plasmodium falciparum (Bell, et al., Biochem. Pharmacol., 48:495-503, 1994; Reddy, et al., Mol. Biochem. Parasitol., 73:111-121, 1995).

Amino acid sequence mutants falling into the range of the cyclophilin defined in the present invention include one or more substitution, insertion, or deletion mutants. These may be prepared by causing nucleotide site-directed mutagenesis in the DNA encoding a wild type cyclophilin through cassette mutagenesis or other well-known techniques to construct DNA fragments encoding mutants, and expressing the recombinant DNA fragments through cell culturing. Amino acid sequence mutants are characterized by expected mutation properties. Typically, the mutants exhibit biological activity principally identical to that of naturally occurring proteins. Mutagenesis techniques capable of introducing substitution mutation into a predetermined site of the DNA whose sequence is analyzed are well known. To this end, for example, M13 primer mutagenesis may be used. Selection of mutants resorts to assaying activities of the cyclophilin proteins. For instance, isomerase activity can be tested by use of techniques well known in the art (Fischer et al., Biomed. Biochim. Acta 43:1101-1111, 1984; Harrison et al., Biochem. 29:1684-1689, 1990). The cyclophilin proteins may be assayed for association with CsA or other immunoregulators. Amino acid substitution is typically targeted to a single residue. Insertion is usually performed with a sequence ranging from 1 to 20 amino acid residues, but a longer insertion is possible. In a typical deletion mutation, 1 to 30 residues are deleted. In some cases, a longer sequence, such as a domain, may be deleted.

For the purpose of the present invention, muteins show formal biological activity identical to that of naturally occurring proteins. However, muteins in which characteristics of cyclophilin are altered, if necessary, may be selected. In another case, muteins or mutant proteins which show altered biological activity of cyclophilin may be designed. For instance, putative residues involved in isomerase activity may be altered. The residues Lys31, Arg55, Gln63, Glu81, Asn102, Trp121 and His126 of human CypA, which are suggested to be brought into contact with CsA, may be altered.

Cyclophilin genes can be isolated using various techniques known in the art. For instance, mRNAs isolated from organisms are reverse-transcribed to give cDNA or expression libraries by use of a typical technique, such as PCR or Southern blotting analysis. The obtained DNA can be cloned into a vector, such as pBR322, pUC19 or T vector. Once being identified, a cyclophilin-encoding DNA sequence can be cloned into a suitable expression vector, for example, an E. coli-derived plasmid (e.g., pET3A, pBluescript or pUC19), a Bacillus subtilis-derived plasmid (e.g., pUB110, pTB5 or pC194), a yeast-derived plasmid (e.g., pSH19 or pSH15), a bacteriophage (e.g., lamda phage), an animal virus (e.g., retrovirus) or an insect virus (e.g., baculovirus). The recombinant vector thus obtained is introduced into a suitable host by using a standard technique for transformation and phage infection. For instance, a calcium chloride method may be applied for the transformation of E. coli (S. N. Cohen, Proc. Natl. Acad. Sci., USA, 69:2110, 1972). The transformation of bacillus may resort to the protocol described in the literature (S. Chang, et al., Molecular and General Genetics, 168:111, 1979). The lithium acetate or spheroplast method disclosed in the literature (Parent, et al., Yeast, 1:83-138, 1985) may be used to transform yeast. The technique described in Virology, 52:456, 1973 is useful for the case of animal cells. The protocol disclosed in Biotechnology, 6:47, 1988 enables baculovirus to transform insect cells. Transformants are cultured according to standard methods suitable for the host cells used. When E. coli is cultured, for example, cells are grown to stationary stage at 30-40° C. in LB media. From the culture of the transformants, for example, cultured cells or culture solution, cyclophilin can be obtained through extraction, isolation and purification processes. For the isolation and purification of cyclophilin from cell cultures or extracts, various techniques can be employed, the techniques exploiting solubility differences such as salt precipitation and solvent precipitation, or molecular weight differences such as dialysis, ultrafiltration, gel filtration and SDS-polyacrylamide gel electrophoresis, or charge differences such as ion exchange column chromatography, or hydrophilicity differences such as reverse phase high performance liquid chromatography, or isoelectric point differences such as isoelectric point focusing electrophoresis.

The term “antioxidants” as used herein means materials scavenging biologically active ROS or other reactive oxygen species (O₂.⁻, H₂O₂, HO., HOCl, pheryl, pheroxyl, pheroxynitryl, and alkoxyl) or catalytically converting free radicals or other reactive oxygen species into weaker reactive species. When being added to cell cultures or assay reaction, the cyclophilin protein of the present invention either removes ROS or decreases ROS to a detectable extent.

In accordance with another aspect, the present invention encompasses a pharmaceutical composition for reducing CsA- or CsA analogue-induced cytotoxicity by the overexpression of cylophilin with PPIase activity in transplanted cells, comprising a recombinant expression vector capable of expressing cyclophilin in the amount sufficient to reduce CsA- or CsA analogue-induced cytotoxicity in transplanted cells.

25 or more CsA amino acid analogues are known (Traber R., HELVETICA ACTA, 70, 13, 1987). PCT Publication Nos. WO 98/28328, WO 98/28329 and WO 98/28330 disclose CsA analogues in which the amino acid residue at position 3 is modified with sarcosin. U.S. Pat. No. 5,318,901 and EP 0 414 632 B1 disclose CsA analogues whose amino acid reside at position 8 is modified. An iso-CsA acid addition salt having a modified amino acid residue at position 1 is suggested in PCT Publication No. WO 93/17039. All of these analogues exhibit immunosuppressive activity like CsA, and their toxicity is the target to which the present invention can be applied. The term “toxicity” as used herein means that deleterious cell death or muscle differentiation blockage is caused by the treatment with CsA or its analogues following tissue or cell transplantation.

The overexpression of cyclophilin in transplanted cells can be achieved through gene therapy. In accordance with an embodiment of the present invention, a general protocol for gene therapy is executed in which retrovirus or adenovirus and virus vectors, all carrying exogenous genes, are infected into desired target cells. In another embodiment of the present invention, gene therapy is carried out by use of transformed cells. In detail, a cyclophilin gene is transfected into cells isolated for transplantation, and high-producing cells are selected from among the transformants and used for gene therapy. The gene therapy according to the procedure satisfies the requirements for a suitable transporter including high efficiency of transmission, stable replication of foreign DNA, appropriate/regulated expression and adequate safety over the time of transfer. A clinical protocol of gene therapy is well known in the art and may be found in the teaching disclosed in E. H. Oldfield, Human Gene Therapy 4:39-69 (1993). A human gene therapy protocol may be obtained from the Office of Recombinant DNA Activities of the National Institutes of Health in Bethesda Md.

Transfection may be defined as the process of introducing polynucleotides which can be expressed (e.g., gene, cDNA or mRNA) into cells. Success in expressing a polynucleotide anchored in cells results in the production of the protein encoded by the polynucleotide in the cells. In an embodiment, the present invention employs a recombinant expression vector carrying a nucleic acid sequence encoding cyclophilin, so as to transfer a DNA encoding cyclophilin into the cells to be transplanted. Cells are transfected with the recombinant expression vector under such a condition as to express cyclophilin. Overexpression of cyclophilin is induced in cells. In an embodiment of the present invention, there is provided cells for use in transplantation which are resistant to CsA or its analogues by overexpression of cylophilin. In this regard, an embodiment of the present invention provides a method for preparing the said cells resistant to CsA or its analogues, comprising the steps of introducing a cyclophilin-encoding gene into a vector to give a recombinant expression vector, transfecting the recombinant expression vector into the cells, culturing the cells, and separating, from the culture, cells in which cyclophilin is over-expressed.

As used herein, the term “vector” means a nucleic acid molecule which is able to carry a cyclophilin gene and may comprise a plasmid, a cosmid, or a virus vector. Vectors may be self-replicating or incorporated into host DNA. Vectors may contain cyclophilin DNA sequences in such suitable forms as to be expressed in host graft cells. Preferably, the recombinant expression vector comprises one or more control sequences operably linked to the cyclophilin nucleic acid sequence. The control sequences may include promoters, enhancers, and/or other expression control components (e.g., polyadenylation signal). The control sequences not only control the constitutive expression of the nucleotide sequence but also include tissue-specific regulation and/or inductive sequences. The design of expression vectors may be determined by such factors as host cells to be transfected, desired expression level, and the like. The recombinant expression vector of the present invention may be designed to cause overexpression of cyclophilin in transfected cells. In accordance with an embodiment of the present invention, the recombinant expression vector may optionally direct nucleic acid expression in specific types of cells. For instance, tissue-specific regulation components are used to control the expression of nucleic acids.

Suitable for gene therapy are virus vectors whose illustrative, but non-limiting examples include replication-deficient retroviruses, adenoviruses and adeno-related viruses. Virus vectors must satisfy the following criteria: (1) vectors can be transfected into desired host cells and thus, virus vectors with an appropriate host range must be selected; (2) the transferred genes must be conserved for an appropriate period of time and expressed; and (3) vectors must be stable to hosts. Other virus vectors which can be used for the transfer of genes into cells include retroviruses such as moloney murine leukemia viruses (MMLV); papovaviruses such as JC, SV40, polyoma, adenovirus and the like; Epstein-Barr viruses (EBV); papiloma viruses such as bovine papiloma virus type I; vaccinia and poliovirus; and other human and animal viruses.

Alternatively, non-virus and non-protein vectors, virus vectors (e.g., adenovirus, herpes virus, vaccinia, retrovirus, etc.), liposomes encapsulating DNA, lipid transfer vehicles, and naked DNA may be used as transport vehicles.

Host expression systems are well known in the art and are used in accordance with an embodiment of the present invention. Any mammalian DNA sequence may be used as a promoter if it can associate with a mammalian RNA polymerase and trigger the downstream transcription of a cyclophilin-encoding sequence into mRNA. Usually, a promoter has a transcription start site adjacent to the 5′-end of a coding sequence and a TATA box that is typically located 25-30 base pairs upstream of the transcription start site. It is believed that the TATA box orders RNA polymerase II to synthesize RNA at a certain position. A mammalian promoter typically comprises an upstream promoter component 100-200 base pairs upstream of a TATA box. The upstream promoter component determines a transcription initiation rate and can operate in either directions. Particularly, a promoter derived from a mammalian virus gene is suitable as a mammalian promoter. The reason is that virus genes are usually highly expressed and have a wide range of host cells. Examples include SV40 early promoters, mouse mammary tumor virus LTR promoters, adenovirus major late promoter, and herpes simplex virus promoters.

A cyclophilin gene may be operably ligated to a promoter which is very precisely regulated like a T7 expression vector (Rosenberg, et al., Gene, 56:125-135, 1987). A potent vector can be prepared by identifying simple restriction targets near both ends of the cyclophilin gene and a suitable restriction target near the promoter on a vector and inserting the cyclophilin gene into the vector in such a way that the gene is under the transcriptional and translational control of the promoter. Alternatively, a cyclophilin gene can be over-expressed by setting up a potent ribosome binding site upstream of the gene (Shine and Dalgarno, Proc. Natl. Acad. Sci. USA, 71:1342-1346, 1974).

Typically, the transcription termination and polyadenylation sequences recognized by mammalian cells are in a control region located at 3′ of a termination codon and thus, are adjacent to the coding sequences, like the promoter components. The 3′ end of a mature mRNA is formed by site-specific post-translation digestion and polyadenylation. Transcription terminators and polyadenylation signals may be exemplified by those derived from SV40.

Introduction of foreign nucleic acids into the cells to be transplanted is well known to those skilled in the art and various introduction techniques may be employed according to host cells used. The introduction usually resorts to dextran-mediated tranfection, electroporation, calcium phosphate precipitation, polybrene-mediated transfection, protoplast fusion, encapsulation of polynucleotide into liposomes, and microinjection of DNA into the nucleus.

Separated cells can be transplanted using standard techniques concerning the separation and transplantation of tissues or organs. Non-self cells are suitably matched according to cell types and transplanted using an immunosuppressant such as cyclosporin. Human cells are used and typically, parenchymal cells, that is, organ cells which functionally work are used. Examples of the organs include liver, pancreas, intestine, uroendothelial cells (including regeneration and uroepithelial structures), cells found in mammary tissues, soft tissues, and endocrine tissues. Also, tissues which are fundamentally of structural function, such as cartilage (cartilage cells, fibroblast), tendon (tendon cells) and bone (bone cells) may be used. The present invention encompasses myogenic cells selected from among myoblasts, myotubes, and young muscle fiber cells. The cells may be normal ones or genetically manipulated ones so as to exhibit normal or additional functions.

The composition of the present invention may further comprise an antioxidant in addition to the above-stated effective components. All kinds of antioxidants known to remove CsA-induced ROS can be used, exemplified by catalase, DFOM (deferoxamine mesylate) and Trion (4,5-dihydroxy-1,3-benzene-disulfonic acid) but are not limited thereto.

Various techniques may be used to administer the pharmaceutical composition of the present invention. For instance, the composition may be administered via parenteral routes by local injection, intramucosal injection or injection into the coelom.

Effective DNA amounts of the recombinant expression vector contained in the pharmaceutical composition of the present invention typically range from about 0.005 to about 50 mg/kg and preferably from about 0.05 to about 5 mg/kg. As generally recognized, the amount range may vary with various factors such as administration methods, target cells, expression levels and the like.

The composition may comprise pharmaceutically acceptable vehicles including all physiological solutions. The vehicles comprise a dispersion medium, a coating agent, an antibacterial agent, an antifungal agent, an isotonic and absorption retardant and the like.

Based on the finding that transplanted cells that have survived pre-exposure to CsA not only reversibly proliferates and differentiates but also are resistant to higher concentrations of CsA, the present invention provides a method for preparing cells for use in transplantation that is resistant to CsA or its analogues, comprising the steps of culturing the cells in a medium following treatment with CsA or its analogues, and recovering viable cells from the culture. Culturing of the cells can be performed according to conventional methods well known in the art. For example, cells are maintained in 100 ml petri dishes containing a proliferation medium supplemented with 10% (v/v) donor calf serum and an antibacterial agent while refreshing the medium every other day. Then, when occupying about 80% of the dish area, the cells are aliquoted into new dishes in a ratio of 1:3.

Cultured cells can be transplanted through suspension, capsules, adsorption to microspheres, or biodegradable or non-biodegradable polymeric fibers (Hansen, et al., Austin, Tex.: R. G. Landes, 1993; 96-106). In an embodiment, calcium alginate capable of forming ionic hydrogel, or other polymers are used to encapsulate cells. Hydrogel may be prepared by crosslinking the anion salts of alginic acid with calcium cations. An alginate solution is mixed with the cells to produce an alginate suspension. Before being hardened, the suspension may be directly injected into patients.

Polymeric materials are combined with cells for transplantation to form hydrogel. Hydrogel comprises natural or synthetic organic polymers which are crosslinked through covalent, ionic and/or hydrogen bonds to form a three-dimensional open-lattice structure which entraps water molecules therein. Examples of the materials used for the formation of hydrogel include polysaccharides such as alginate, polyphosphagin, polyacrylate, and Pluronics™ or Tetronics™. The last two polymers are crosslinked by temperature or pH. Also, proteins such as fibrin, polymer such as polyvinylpyrrolidone, hyaluronic acid and collagen are used as well.

In a preferable embodiment, a cell suspension is obtained as follows. A polymer is dissolved at such a high concentration as to form a hydrogel, for example, in the case of alginate, at 0.5-2 weight % and preferably at 1 weight % in an aqueous solution of physiological pH and preferably in a 0.1 M potassium phosphate solution. Isolated cells for transplantation are suspended at a concentration of 1-5×10⁷ cells/ml and most preferably at a concentration of 1-2×10⁷ cells/ml in a polymer solution.

Matrixes for cell transplantation may be also prepared. These matrixes must have sufficiently large surface areas and be exposed to sufficiently rich nutrients so that cells proliferate and differentiate in advance of transfer into blood vessels. Cells may be sown on a matrix which is then transplanted. Alternatively, a matrix is first transplanted to a desired region and then cells are injected onto the matrix. The matrix suitable for the present invention must be a flexible, non-toxic and injectable porous template so as for the cells to grow in blood vessels. In a preferable embodiment, the matrix is prepared from a bioabsorptive, biodegradable, synthetic polymer such as polyanhydride, polyorthoester, polylactic acid, polyglycol, a copolymer thereof, or a blend thereof. Examples of suitable materials include ethylene vinyl acetate, polyvinyl alcohol derivatives, Teflon, and nylon. The absorption of cells onto the polymer can be improved by coating the polymer with basal membrane components, agar, agarose, gelatin, gum Arabic, collagen I, II, III, IV or V, fibronectin, laminin, of glycosaminoglycan.

In accordance with the present invention, the cells of the present invention which are induced to overexpress cyclophilin in advance can survive against the toxicity caused by treatment with CsA or its analogues and thus can be transplanted at a high success rate. In an embodiment of the present invention, there are provided myoblasts that are induced into the overexpression of CsA. The myoblasts of the present invention can be useful in treating diseases related to muscle degeneration or weakness, such as myopathy.

The results of the following examples are expressed as mean±standard deviation values of data obtained from at least 3 independent experiments and statistically analyzed using Student's t-test and one-way ANOVA. Unless specifically stated, the significance level was set at P<0.05.

Materials used in the following examples were obtained as follows. Dulbecco's modified Eagle's Medium (DMEM), Dulbecco's modified Eagle's Medium/F-12 (DMEM/F-12), fetal bovine serum, donor calf serum, lipofectin and G418 were purchased from GIBCO/BRL(Grand Island, N.Y., USA). Ham's F-10 media, collagenase, L-phenylephrine, catalase, 2′,7′-dichlorofluorescein diacetate (DCF), 3,3′-dihexyloxacarbocyanine iodide, DiOC6[3], 4,5-dihydroxy-1,3-benzene-disulfonic acid (Tiron) and deferoxamine mesylate (DFOM) were purchased from Sigma Chemical Co.(St. Louis, Mo., USA). CsA, FK506, ascomycin and caspase 3 suppressant II(DEVD) were purchased from Calbiochem (San Diego, Calif.). Antibodies against myogenin, MRF4, β-actin and c-myc were purchased from Santa Cruz Biotechnology (Santa Cruz, Calif., USA). A monoclonal antibody specific to CypA was purchased from Upstate Biotechnology (Lake Placid, N.Y., USA).

### EXAMPLE 1

### Inhibition of Calcineurin Phosphatase Activity by CsA, FK506 and Ascomycin

Because CsA inhibits the phosphatase activity of calcineurin that is transiently increased during differentiation, some researchers have suggested that CsA-induced differentiation block might be caused by the inhibition of the phosphatase activity of calcineurin (Abbott, K. L. et al., Mol. Biol. Cell. 9, 2905-2916, 1998; Friday, B. B. et al., J. Cell. Biol. 149, 657-666, 2000). The present inventors examined the effects of immunosuppressants, CsA, FK506 and ascomycin on the calcineurin phosphatase activity.

First, the concentration of the immunosuppressants at which the calcineurin phosphatase activity could be maximally inhibited was determined. Cardiac myoblasts of H9c2 rats (ATCC, Manassas, Va., USA) were maintained in a DMEM/F-12 medium supplemented with 10% (v/v) donor calf serum and antibiotic mixture (penicillin G, streptomycin and amphotericin B) (Proliferation Medium, PM). Muscle differentiation was induced in a DMEM/F-12 supplemented with 1% (v/v) horse serum (HS) (Differentiation Medium, DM), when the myoblasts were confluent. Following independent addition of CsA, FK506 and ascomycin to the DM, the myoblasts of H9c2 rats were cultured for 2 days to measure the phosphatase activity of calcineurin. The DM was refreshed every other day.

The phosphatase activity of calcineurin was measured as follows. After being collected in 1 ml of trypsin-EDTA, the myoblasts were pelleted and lysed in 100 ml of a calcineurin assay buffer (BioMol, Plymouth Meeting, Pa., USA). Using BioMol QuantizymeTM Assay System AK-804, the phosphatase activity of calcineurin was measured as instructed in the protocol provided by the manufacturer. According to the instruction, the phosphatase activity of calcineurin was measured on a microtiter-plate reader at OD 620 nm by detecting free-phosphate released from calcineurin-specific RII phosphopeptide. The measured calcineurin phosphatase activity was standardized by the values measured in serum-starved cells (SF).

In order to induce the phosphatase activity of calcineurin with L-phenylephrine (PE), serum-free DMEM/F-12 was deprived of cytotoxic serum for 4 hours, followed by treating the media with PE for 12 hours. The immunosuppressants were added to the media 30 min before the addition of PE.

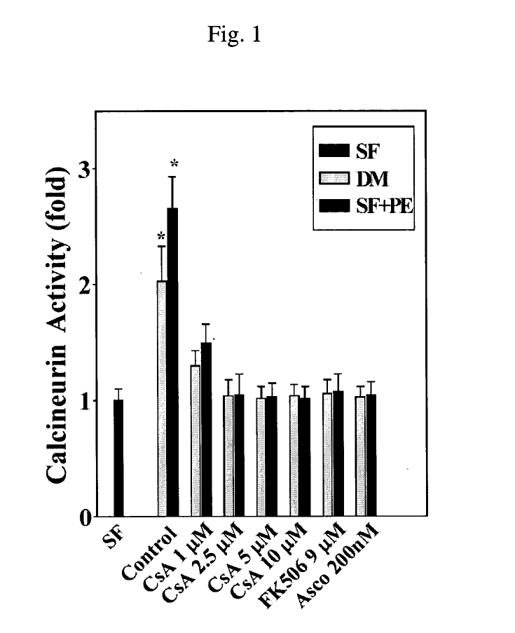

The phosphatase activities of calcineurin measured above are shown in FIG. 1. Each value in FIG. 1 represents a fold activity compared to the basal calcineurin phosphatase activity measured 12 hours after serum removal.

As seen in FIG. 1, calcinuerin phosphatase activities induced by DM and PE in control were increased about 2.1 fold and 2.7 fold respectively. Maximal inhibitory effects on the increased calcinerin phosphatase activities were observed at 2.5 μM of CsA, 1 μM of FK506 and 20 nM of ascomycin. However, 9 μM of FK506 and 200 nM of ascomycin were used in subsequent experiments to assure the inhibition of the induced calcineurin phosphatase activity. The basal activities were not influenced by the drugs. The inhibitory effects of the drugs on the calcineurin phosphatase activities increased by PE were regarded as positive controls. Consequently, CsA, FK506 and ascomycin could completely inhibit the calcineurin phosphatase activity induced during muscle differentiation, but had no influence on the basal activity.

### EXAMPLE 2

### Effects of CsA, FK506 and Ascomycin on Muscle Differentiation and Apoptosis

2-1. Effect of CsA on Muscle Differentiation of Rat Cardiac Myoblasts

Effects of CsA, FK506, ascomycin and SDZ NIM811 on the differentiation of H9c2 rat cardiac myoblasts were investigated. SDZ NIM811 is a CsA analogue lacking calcineurin inhibitory activity.

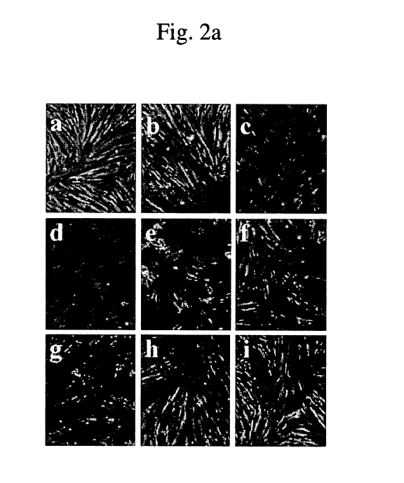

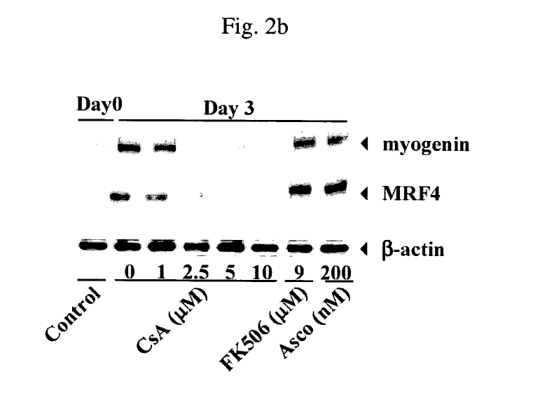

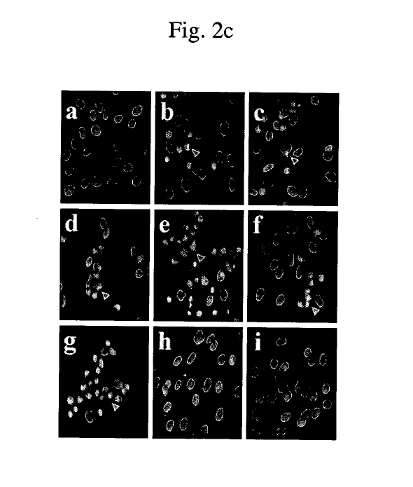

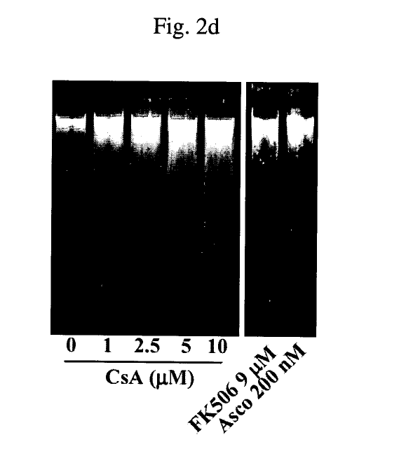

After being cultured for 72 hours in DM in the respective presence of CsA, FK506, ascomycin and SDZ NIM811, H9c2 rat cardiac myoblasts were observed for morphological changes during muscle differentiation. The results are given in FIG. 2a. As a control, myoblasts were cultured in the absence of the drugs. Before use, the myoblasts were cultured to a cell confluency of about 70%.

It is apparent from FIG. 2a that while treatment with CsA or SDZ NIM811 blocks the myoblast differentiation with concomitant reduction of cell populations, treatment with FK506 or ascomycin results in no morphological differences in comparison to the control.

In addition, the protein expression levels of the myogenin and MRF4, early and late myogenic markers, were determined by Western blot analysis. To this end, myoblast extracts treated with the drugs were separated on SDS-PAGE and transferred onto a nitrocellulose membrane which was then blocked and reacted with predetermined primary antibodies (polyclonal myogenic antibody and polyclonal MRF4 antibody (Santa Cruz, Calif., USA) and then with a secondary antibody (anti-rabbit IgG-HRP conjugate with a chemiluminescent reactive enzyme able to detect the primary antibodies) (Santa Cruz, Calif., USA). Finally, the specimens were detected by enhanced ECL-plus chemiluminescence (Amersham Pharmacia Biotech, Piscataway, N.J., USA). Non-myogenic β-actin protein was used as a loading control. The results are shown in FIG. 2b.

As seen in FIG. 2b, CsA and SDZ NIM811 exhibited partial suppression at 1 μM and complete suppression at higher concentrations. However, FK506 and ascomycin were found to exert no influence.

2-2 Effect of CsA on Apoptosis of Rat Cardiac Myoblasts

Effects of CsA on apoptosis were measured with resort to in-situ TUNEL assay and DNA fragmentation analysis.

H9c2 myoblasts were cultured in a DM for 72 hours in the presence of CsA, FK506, ascomycin or SDZ NIM811, spread on a slide and fixed with 1% para-formaldehyde. TUNEL analysis was performed using ApopTag Flurescein In situ Apoptosis Detection kit (Intergen, Purchase, N.Y.). The results are given in FIG. 2c where arrows indicate TUNEL positive cells.

Once cells undergo apoptosis, the DNA is digested into fragments diferring by 180-200 bp by endonuclease. Separation on agarose gel shows a DNA band pattern like a ladder. DNA fragmentation aiming to identify the DNA laddering pattern was performed as follows. H9c2 myoblasts were cultured in DM for 72 hours in the presence of CsA, FK506, ascomycin or SDZ NIM811 and lysed in a lysis buffer (50 mM Tris-HCl(pH 7.5), 100 mM NaCl, 20 mM EDTA and 0.5% SDS). The cell lysates thus obtained were treated with 50 μg/ml RNase A at 37° C. for 2 hours and then with 500 μg/ml proteinase K at 55° C. for 2 hours. DNA was extracted by a phenol/chloroform method and precipitated in 70% (v/v) isopropanol. After being centrifuged at 15,000 rpm for 15 min, the precipitates were dissolved in a TE buffer (10 mM Tris-HCl(pH 8.0), 1 mM EDTA). DNA was analyzed on 1.5% agarose gel by electrophoresis, and the result is given in FIG. 2d.

As apparent from FIGS. 2c and 2d, CsA and SDZ NIM811-induced apoptosis is dose dependent, but FK506 and ascomycin are not related to apoptosis.

2-3 Effect of CsA on Muscle Differentiation and Apoptosis of Rat Limb Skeletal Muscle

H9c2 myoblasts, the muscle cells of rat heart, showed characteristic morphology following differentiation. For comparison, myoblasts taken from the limb of a Spraque-Dawley rat 4 days after birth were used to investigate the cell death and muscle differentiation effects of CsA, FK506, ascomycin and SDZ NIM811.

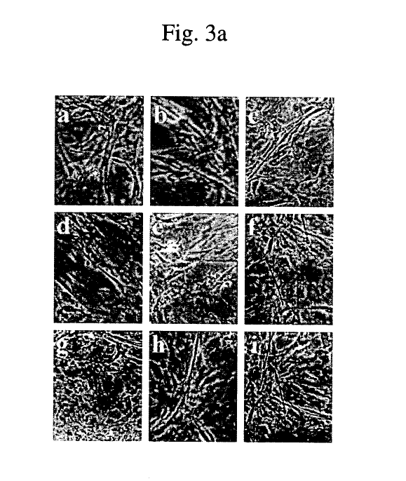

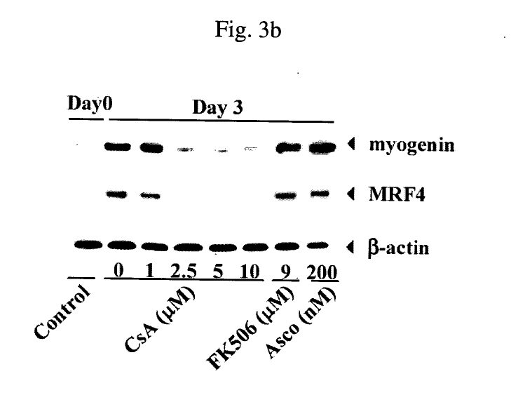

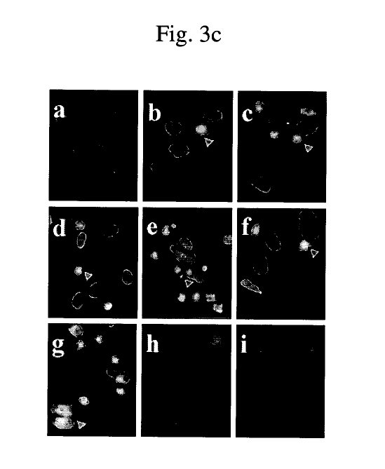

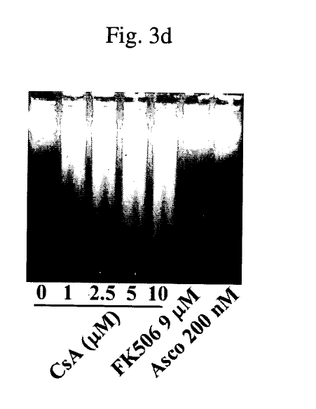

A muscle tissue separated from the limb of a 4-day-old Spraque-Dawley rat was minced and cultured in a serum-free DMEM/Ham's F-10 (1:1, v/v) for 24 hours at 37° C. in the presence of collagenase (2000 U). After a low speed centrifugation, the cells thus harvested were resuspended in DMEM/Ham's F-10 supplemented with 10% fetal bovine serum and 1 % antibiotics (penicillin G and amphotericin B). These cells were transferred in a high density at 37° C. into petri dishes. Cells were separated again and incubated for 72 hours in DMEM/Ham's F-10 (supplemented with 1% horse serum) in the presence of CsA, SDZ NIM811, FK506 and ascomycin. Morphological analysis, Western blot, TUNEL assay and DNA fragmentation were conducted in the same manner as in Examples 2-1 and 2-2. The results are shown in FIGS. 3a and 3d.

It is apparent from FIGS. 3a to 3d that myoblasts of rat limb skeletal muscle and H9c2 myoblasts respond in a similar manner.

In contradiction to prior research results (Abbott, K. L. et al., Activation and cellular localization of the cyclosporine A-sensitive transcription factor NF-AT in skeletal muscle cells, Mol. Biol. Cell. 9, 2905-2916, 1998; Friday, B. B. et al., Calcineurin activity is required for the initiation of skeletal muscle differentiation, J. Cell. Biol. 149, 657-666, 2000), the results obtained in the above examples indicate that the induction of calcineurin phosphatase activity during muscle differentiation is not indispensable for muscle differentiation.

2-4 Effect of CsA on Muscle Differentiation and Apoptosis During Muscle Differentiation

Since the cells that have evaded muscle differentiation blockage in the early stage (myogenin expression), progress into the late differentiation stage (MRF4 expression), it is expected that the cytotoxicity of CsA to myoblasts will be diminished in the late stage. To confirm this, the CsA blockage of muscle differentiation and apoptosis was measured during the muscle differentiation stages.

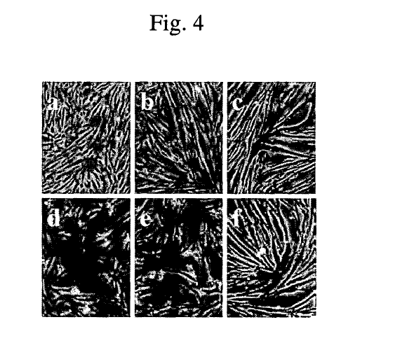

Cells were cultured in DM for 24, 48 and 72 hours, and their morphological changes were observed. The results are shown in FIGS. 4a, 4b and 4c. Later, cells were treated with 10 μM CsA, then, cultured in DM for additional 72 hours. The morphological changes thus obtained are shown in FIGS. 4d, 4e and 4f.

The photographs of FIG. 4 reveal that toxic effects are found in the cells that have been cultured for 24 and 48 hours in DM (those that have not entered the myotube formation stage yet) (a, b, c and e), while the cells differentiated into myotubes in 72 hours (c and f) experienced no change in cell population and morphology.

### EXAMPLE 3

### Induction of Apopotosis by CsA through ROS Generation

3-1 ROS Generation by CsA and Relation between Antioxidant and ROS Level

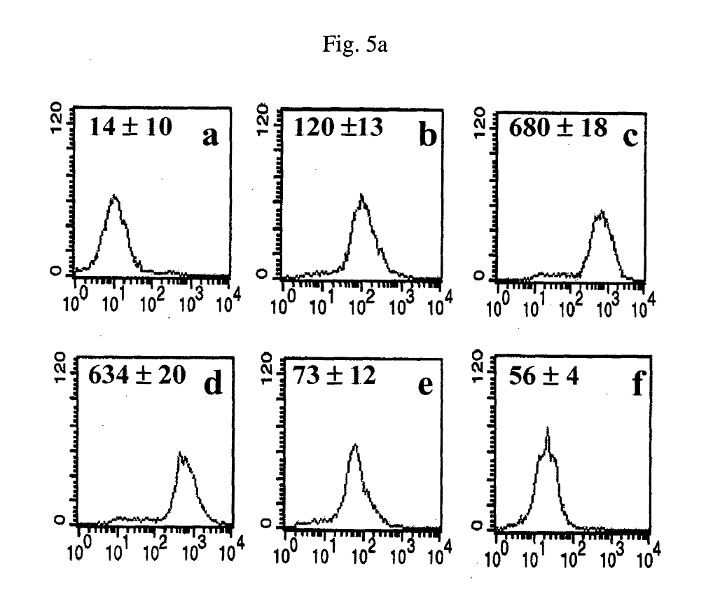

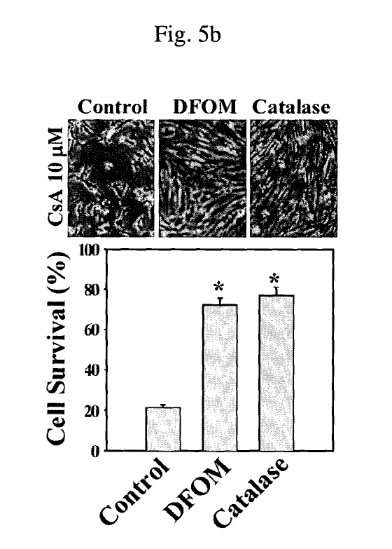

H9c2 myoblasts were cultured for 24 hours in PM and in DM, and then in the presence of 10 μM DCF(2′,7′-dichlorofluorescein). After being harvested, the cells were washed once with PBS and resuspended in 800 ml of PBS. The cellular levels of ROS generated were measured by flow cytometry (FACS), there values were regarded as controls. Next, the PM was converted into DM in which cells were incubated for 24 hours in the presence of 10 μM CsA or 10 μM SDZ NIM811. ROS levels were analyzed by FACS. Separately, 0.4 mM deferoxamine mesylate (DFOM) or 2000 units/ml catalase was added to DM, incubated for 30 min, then, 10 μM CsA was added and the cells were incubated for another 24 hrs. ROS levels were measure as the above. The results are given in FIG. 5a where numerals indicate mean±standard deviation values of the ROS levels in the cells.

As shown in FIG. 5a, the differentiation-related ROS levels of the cells cultured for 24 hours in DM in the presence of CsA and NIM811 amounted to 680±18 and 634±20, respectively, being at least 5-fold greater than those of the cells cultured in the absence of the drugs (c and d of FIG. 5a). In the co-presence of CsA/DFOM and CsA/catalase, the ROS levels were measured to be as low as 73±12 and 56±4, respectively, implying that antioxidants restrict the generation of ROS (e and f of FIG. 5a).

3-2 Cell Protection Effect of Antioxidants

To determine if antioxidants protect cells from CsA-induced apoptosis, H9c2 myoblasts were cultured in the presence of 10 μM CsA, 0.4 mM DFOM/10 μM CsA, and 2000 units/ml catalase/10 μM CsA, with their morphological changes being monitored. Percentages of cell survival were measured, as well. The results are given in FIG. 5b.

As apparent from FIG. 5b, antioxidants can protect cells from CsA-induced apoptosis.

### EXAMPLE 4

### Role of Bax/Mitochondria Membrane Potential/Caspase 3 in CsA-Induced Apoptosis

4-1 Western Blotting for Analysis of the Apoptosis-Accelerating Protein Bax

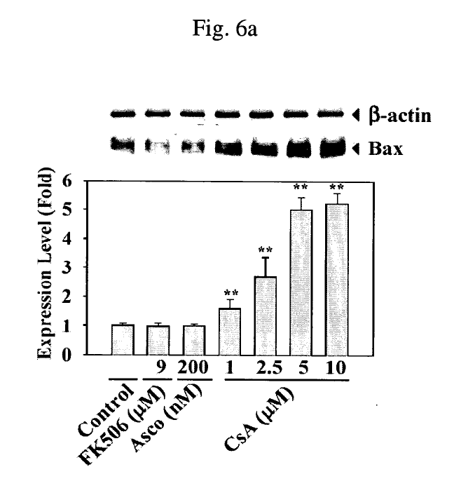

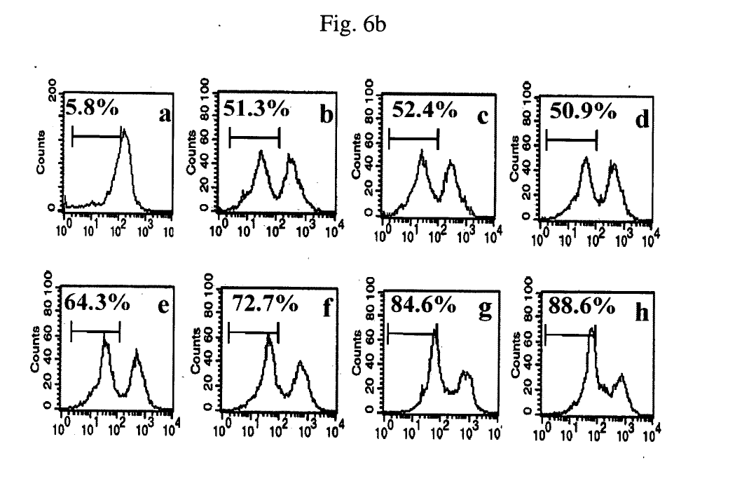

H9c2 myoblasts were cultured in DM in the presence of 9 μM FK506, 200 nM ascomycin, or 1-10 μM CsA or in the absence of any of the drugs (control), and were subjected to Western blotting analysis in the same manner as in Example 2-1. The results are shown in the upper part of FIG. 6a. β-actin is used as a loading control.

After being scanned, the Western blot analysis bands were quantified by Bio-Rad imaging densitometer. The results are shown in the lower part of FIG. 6a. In the histograms, the bars express the Bax levels of the samples as mean±standard deviation values of the fold numbers compared to those of the control.

As seen in FIG. 6a, treatment with CsA increases Bax expression in a dose-dependent manner while FK506 and ascomycin do not affect the expression of Bax.

4-2 Measurement of Mitochondria Membrane Potential (ΔΨm)

H9c2 myoblasts were cultured for 48 hours in DM in the presence of 9 μM FK506, 200 nM ascomycin, or 1-10 μM CsA and then for 30 min at 37° C. in the presence of 40 nM 3,3′-dihexyloxacarbocyanine iodide (DiOC6[3]), and were washed with PBS, following which their mitochondrial membrane potentials were measured by use of flow cytometry. H9c2 myoblasts cultured in PM and in DM in the absence of the drugs were measured for ΔΨm (control). The results are shown in FIG. 6b. In the FACS plots of FIG. 6b, the second peaks and the first peaks represent viable cells with high DiOC6[3] contents and dead cells with DiOC6[3]. Numerals mean percentages of the myoblasts with decreased ΔΨm.

As seen in the FACS profiles, a remarkable decrease of ΔΨm was observed only in DM, and CsA treatment reduced ΔΨm in a dose-dependent manner. However, such reductions were not observed in the case of FK506 or ascomycin.

4-3 Quantitative Analysis of CsA-Induced Apoptosis and Determination of Caspase Contribution

H9c2 myoblasts were cultured in DM for 72 hours in the presence of CsA, FK506 or ascomycin and then dyed with propydium iodide for FACS (fluorescence-activated cell sorting) analysis. Separately, 30 min before the treatment with CsA, cells were treated with 50 μM of the caspase-3 inhibitor DEVD and precultured. Using 20,000 cells obtained from each of the cultures, measurements were made of percentages of cells in each phase of the cell cycle (G₀/G₁, S and G₂/M) and percentages of cells in sub-G₀/G₁ aliquots (that is, apoptotic cells). The results are given in Table 1, below.

TABLE 1
Dose-response effects of CsA on the cell cycle and apoptosis in H9c2
cardiac myoblasts

| | % Cells in each phase of the cell cycle and | | | |
|---|---|---|---|---|
| | apoptotic cells in sub-G₀/G₁ fraction | | | |
| Treatment | % G₀/G₁ | % S | % G₂/M | % Apoptosis |
| CsA 0 μM | 86 ± 3 | 3 ± 1 | 4 ± 1 | 10 ± 2 |
| CsA 1 μM | 63 ± 4 | 5 ± 1 | 3 ± 1 | 21 ± 3^{a} |
| CsA 1 μM + DEVD 50 μM | 76 ± 4 | 4 ± 1 | 3 ± 1 | 15 ± 2^{b} |
| CsA 2.5 μM | 62 ± 4 | 3 ± 1 | 2 ± 1 | 36 ± 4^{a} |
| CsA 5 μM | 44 ± 3 | 2 ± 1 | 1 ± 1 | 55 ± 3^{a} |
| CsA 10 μM | 20 ± 4 | 1 ± 1 | 2 ± 1 | 78 ± 3^{a} |
| CsA 10 μM + DEVD 50 μM | 35 ± 4 | 2 ± 1 | 3 ± 1 | 60 ± 3^{b} |
| FK506 9 μM | 83 ± 5 | 4 ± 1 | 4 ± 1 | 11 ± 3 |
| Ascomycin 200 nM | 85 ± 3 | 2 ± 1 | 4 ± 1 | 10 ± 3 |

Note:
^{a}significance level set at P < 0.05 compared to control (CsA 0 μM)
^{b}significance level set at P < 0.05 compared to cells treated only with CsA at corresponding concentrations

The DNA analysis data by FACS of Table 1 leads to the conclusion that CsA shows dose-dependent apoptotic induction, but FK506 and ascomycin do not, and DEVD cannot completely block apoptotic cell death, implying that caspase-3 may be not a unique mediator of CsA-induced apoptosis.

### EXAMPLE 5

### Antioxidative Action of CypA

To examine if the inhibition of the PPIase activity of CypA by CsA is related to the differentiation blockage induced by drugs and if CypA protein itself provides such a ROS scavenging effect as to alleviate CsA-induced cytotoxicity, first, cells transfected with a CypA were selected.

Preparation of the Wild Type CypA Gene-Transfected Cell CypA/wt

By RT-PCR using the following primers, a wild type CypA cDNA fragment of rat brain was amplified.

(SEQ ID No. 1)

5′-GCAAGCTTACCATGGTCAACCCCACC-3′
(forward primer, HindIII site (underlined) and
Kozak sequence contained).

(SEQ ID No. 2)

5′-GCGGATCCGAGTTGTCCACAGTCGGA-3′
(reverse primer, BamHI site (underlined)
contained).

The amplified wild type CypA cDNA fragment was cloned, together with a c-myc tag fragment containing a stop codon, in pcDNA 3.0, a mammalian expression vector (Invitrogen, Calif., USA). The resulting recombinant expression vector was transfected into H9c2 myoblasts with the aid of Lipofectin as instructed in the protocol provided from the manufacturer. Afterwards, the transfected cells were cultured for one month in the presence of 200 μg/ml G418 to select wild type CypA gene-transfected cells CypA/wt.

Preparation of CypA-PPIase Activity Mutant Cell CypA/R55A

Using one of the following oligonucleotides as a mutagenic primer (mismatching bases underlined), PCR was performed to give a double-stranded PCR product in which an arginine residue was substituted with an alanine residue at position 55. The PCR product was used as a megaprimer in other PCR to produce full-length mutant cDNA in which an arginine residue is substituted with an alanine residue at position 55:

| | |
|---|---|
| 5′-TCCTTTCACGCGATTATTCCA-3′ | (SEQ ID No. 3) |
| 5′-TGGAATAATCGCGTGAAAGGA-3′ | (SEQ ID No. 4) |

The amplified mutant cDNA fragment was cloned, together with a c-myc tag fragment containing a stop codon, in pcDNA 3.0, a mammalian expression vector (Invitrogen, Calif., USA). The resulting recombinant expression vector was transfected into H9c2 myoblasts with the aid of Lipofectin as instructed in the protocol provided from the manufacturer. Afterwards, the transfected cells were cultured for one month in the presence of 200 μg/ml G418 to select mutant CypA gene-transfected cells CypA/R55A.

Preparation of CypA-PPIase Activity Mutant Cell CypA/W121F

Using one of the following oligonucleotides as a mutagenic primer (mismatching bases underlined), PCR was performed to give a double-stranded PCR product in which a tryptophan residue was substituted with a phenylalanine residue at position 121. The PCR product was used as a megaprimer in other PCR to produce full-length mutant cDNA in which a tryptophan residue is substituted with a phenylalanine residue at position 121:

| | |
|---|---|
| 5′-AAGACTGAGTTCCTGGATGGC-3′ | (SEQ ID No. 5) |
| 5′-GCCATCCAGGAACTCAGTCTT-3′ | (SEQ ID No. 6) |

The amplified mutant cDNA fragment was cloned, together with a c-myc tag fragment containing a stop codon, in pcDNA 3.0, a mammalian expression vector (Invitrogen, Calif., USA). The resulting recombinant expression vector was transfected into H9c2 myoblasts with the aid of Lipofectin as instructed in the protocol provided from the manufacturer. Afterwards, the transfected cells were cultured for one month in the presence of 200 μg/ml G418 to select mutant CypA gene-transfected cells CypA/W121F.

5-1 Analysis for CypA Expression by Western Blotting

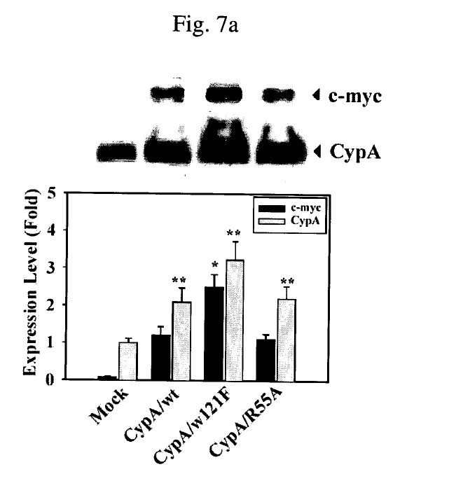

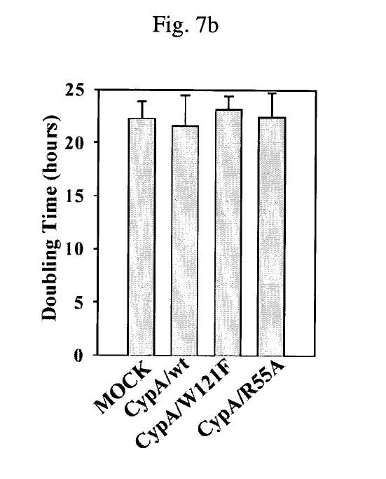

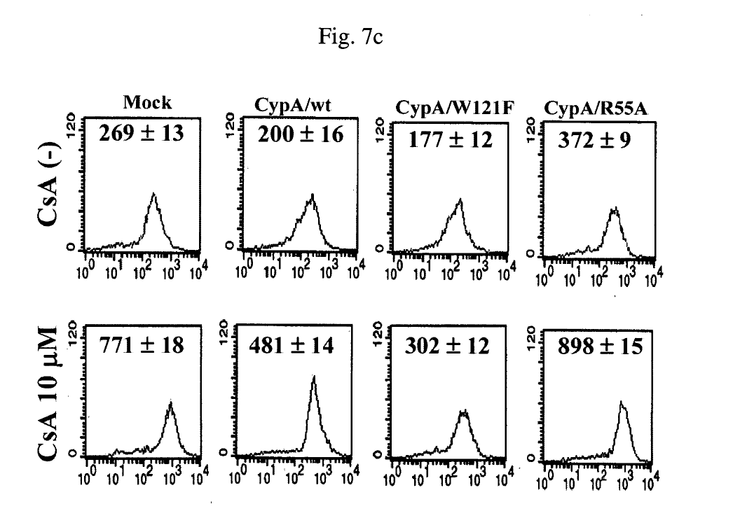

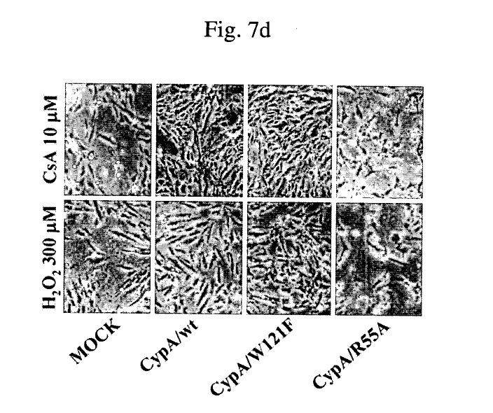

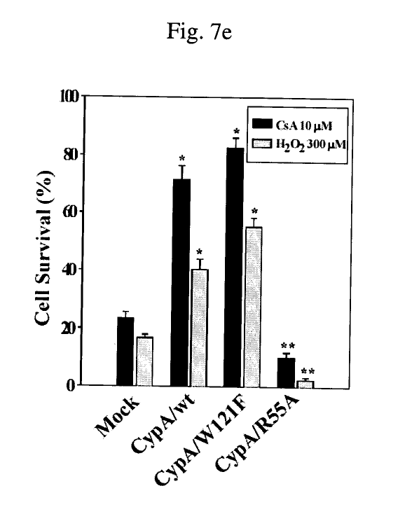

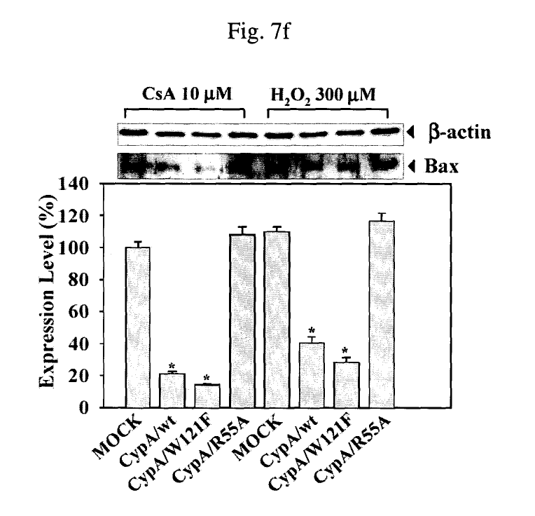

Expression levels of CypA in the cells prepared above, CypA/wt, Cyp/W121F and CypA/R55A, were analyzed by a Western blotting method using a c-myc-specific antibody and a CypA-specific antibody, as in Example 2-1. The Western blotting results are given in FIG. 7a. In the drawing, the upper part shows immunoblots which are quantitatively analyzed by use of Bio-Rad imaging densitometer. In the histograms, bars express the c-myc and CypA levels of the CsA-treated samples as mean±standard deviation values of the fold numbers compared to those of the control.

As seen in FIG. 7a, the expression level of CypA/wt or CypA/R55A is similar to the base expression level attributed to the chromosomal DNA of CypA, but the expression level of CypA/W121F is twice as much.

5-2 Measurement of Doubling Time

The effect of CypA on cell proliferation was examined in terms of doubling time. To this end, the above-prepared cells CypA/wt, CypA/W121F and CypA/R55A were stained with trypan blue and counted every day to measure their doubling time. FIG. 7b shows the results.

As seen in FIG. 7b, the overexpression of CypA, whether wild type or mutants, has no influence on cell proliferation, indicating that CypA does not have a function indispensable for cell proliferation.

5-3 Effect of CypA Overexpression on ROS Generation upon Differentiation Induction in the Presence or Absence of CsA

In DM containing no CsA or 10 μM CsA, CypA/wt, CypA/W121F and CypA/R55A were cultured. These transfected cells were grown at 37° C. in the presence of 10 μM DCF, washed with PBS, resuspended in 800 ml of PBS, and measured for ROS content by FACS analysis. The results are shown in FIG. 7c.

As seen in the FACS plots, CypA/wt reduced the ROS level either in DM containing no CsA or in DM containing CsA. Further, CypA/W121F removed a greater amount of ROS than did CypA/wt, which indicates that the ROS scavenging ability is directly related to the level of available CypA proteins (see Example 5-1). On the contrary, CypA/R55A significantly increased ROS generation, implying the involvement of the PPIase activity in ROS scavenging.

5-4 CsA and H₂O₂ Exposure Effect on Cell Protection from Apoptosis

CypA/wt, CypA/W121F and CypA/R55A were exposed to 10 μM CsA for 72 hours in DM. Separately, the cells were exposed to 300 μM H₂O₂ for 12 hours in DMEM/F-12. Their morphological changes were monitored during the exposure and are shown in FIG. 7d. After exposure to CsA and H₂O₂, viable cells were counted to calculate % cell survival. Here, % cell survival is obtained by dividing pre-exposure cell counts with post-exposure cell counts. Cell survivals are given in FIG. 7e.

It is apparent from FIGS. 7d and 7e that CypA/wt and CypA/W121F protect cells from CsA- or H₂O₂-induced apoptotic cell death, but not from differentiation blockage, and CypA/RS5A increases ROS-induced apoptosis.

5-5 Comparison of Bax Expression Levels

Following the exposure of the transfected cells CypA/wt, CypA/W121F and CypA/R55A to CsA or H₂O₂, Bax expression levels induced by CsA or H₂O₂ were analyzed by Western blotting, as described in Example 5-4. FIG. 7f shows the Western blot results. In the histograms of FIG. 7f, bars express the Bax levels in the samples as percentages compared to the mock-transfected cells.

When being exposed to CsA, CypA/wt and CypA/W121F cells showed inhibited Bax expression, but CypA/R55A cells showed increased Bax expression. Similar results were obtained upon treatment with H₂O₂.

### EXAMPLE 6

### Role of CypA-PPIase Activity in Muscle Differentiation

6-1 Observation of Morphological Changes upon Muscle Differentiation

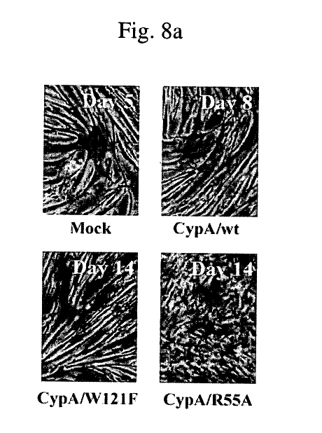

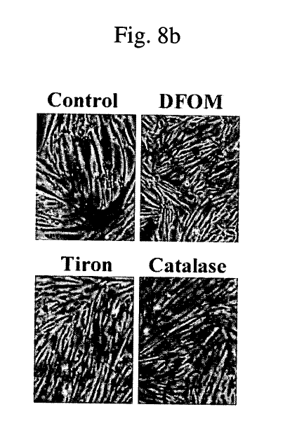

To determine the role of CypA, that is, PPIase, in muscle differentiation, time periods required for the complete differentiation of the three CypA gene-transfected cells, CypA/wt, CypA/W121F and CypA/R55A were measured while they were cultured in DM. In addition, their morphological changes were observed when they were completely differentiated. The results are shown in FIG. 8a.

The photographs of FIG. 8a illustrate that CypA/wt and CypA/W121F cells undergo muscle differentiation in DM, but no traces of differentiation were found in CypA/R55A. Accordingly, PPIase activity is believed to be necessary for muscle differentiation.

Time required for muscle differentiation was 4-5 days for the control (pcDNA3.0-transfected H9c3 cardiac myoblasts (mock)), 8-9 days for CypA/wt cells, and 14-15 days for CypA/W121F cells. From these measurements, it is understood that muscle differentiation is delayed in a CypA dose-dependent manner.

6-2 Effect of Antioxidants on Muscle Differentiation

H9c2 myoblasts were cultured for 5 days in DM containing 0.4 mM of DFOM, 2000 units/ml of catalase, 10 mM of Tiron (4,5-dihydroxy-1,3-benzene-disulfonic acid) to induce differentiation, during which morphological changes were observed. The results are shown in FIG. 8b. The control is the myoblasts which were induced to differentiate in the absence of antibiotics.

The photographs of FIG. 8b describe the blocking activity of DFOM, catalase and Tiron block in muscle differentiation, implying that ROS acts as a trigger of the differentiation process and that the differentiation can be delayed by reducing ROS levels as in CypA/wt and CypA/W121F cells.

### EXAMPLE 7

### Reversibility and Acquired Resistance of Myoblasts Pre-Exposed to CsA

7-1 Induction of CypA Expression by CsA

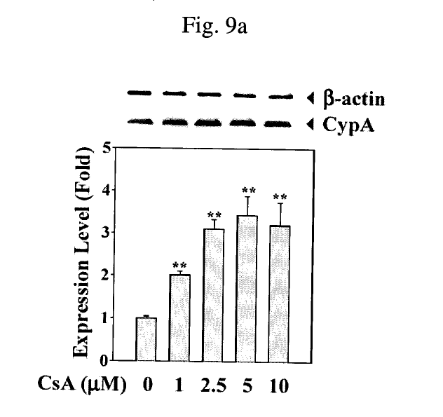

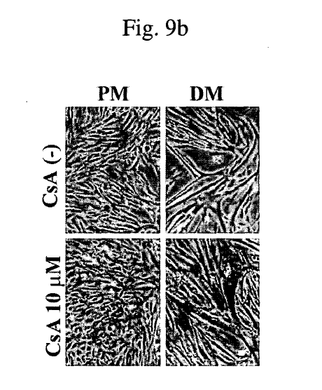

To investigate if CsA could induce CypA expression, H9c2 myoblasts were treated with 0-10 μM CsA for 48 hours in DM. Expression levels of CypA were measured by Western blotting analysis using a CypA-specific antibody and a β-actin-specific antibody. FIG. 9a shows the Western blot results. In the histograms of FIG. 9a, bars represent the expression levels, quantitatively standardized relative to the control which was not treated with CsA.

As seen in FIG. 9a, the expression of CypA protein somewhat decreases at 10 μM CsA but, on the whole, increases proportionally with increasing of CsA (in the range of 1 to 5 μM).

7-2 Resistance and Reversibility Acquirement of Myoblasts

H9c2 myoblasts pre-exposed in DM to 5 μM of CsA, a concentration causing complete differentiation blockage and 55% cell death, were washed with PBS and cultured for 48 and 72 hours in PM or DM, with the observation of morphological changes, to examine if cells could proliferate and differentiate reversibly. These results are given in the upper panels of FIG. 9b.

Separately, H9c2 myoblasts pre-exposed to 5 μM CsA in DM were washed with PBS, cultured for 48 hours in PM, and exposed again to 10 μM CsA in PM for 48 hours or to 10 μM CsA in DM for 72 hours, while monitoring their morphological changes. The results are shown in the lower panels of FIG. 9b.

Photographs of FIG. 9b verify that cells pre-exposed to CsA are able to proliferate and differentiate reversibly upon subsequent replacement in the corresponding media, and also in the media containing a higher CsA concentration. That is, not only do the cells survive the stress of 5 μM CsA in DM, but also the pre-exposed cells acquired resistance so as to proliferate and differentiate even in 10 μM CsA condition to the same extent as the control (treated with no CsA (differentiation control of FIG. 8b)). These findings indicate that pre-exposed myoblasts do not undergo differentiation delay upon subsequent exposure to higher CsA concentrations and thus, the induction of stress proteins including CypA contributes to the acquired resistance.

Taken together, the data obtained in the examples demonstrate that the cyclophilin protein with PPIase activity is very useful as an antioxidant and the overexpression of the cyclophilin protein in transplanted cells makes a great contribution to the success of transplantations.

1. An antioxidant against ROS (reactive oxygen species) induced by cyclosporin A or its analogues, comprising a cyclophilin protein with PPIase (peptidyl-prolyl-cis-trans isomerase) activity.

2. A pharmaceutical composition for preventing cyclosporin A-induced cytotoxicity by the overexpression of cylcophilin with PPIase activity in transplanted cells, comprising a recombinant expression vector which can express the cyclophilin protein in such a sufficient amount as to reduce the toxicity induced by cyclosporin A or its analogues in transplanted cells.

3. The pharmaceutical composition as defined in claim 2, wherein the transplanted cells are myoblasts.

4. A cell for use in the transplantation which is resistant to cyclosporin A or its analogues, wherein a cyclophilin protein with PPIase activity is over-expressed.

5. The cell as defined in claim 4, wherein the cell is a myoblast.

6. A method of preparing cells for use in the transplantation which are resistant to cyclosporin A or its analogues, comprising the steps of introducing a gene encoding a cyclophilin protein with PPIase activity into a vector to construct a recombinant expression vector, transfecting the recombinant expression vector into cells to be transplanted, culturing the transfected cells, and selecting cells in which the cyclophilin with PPIase activity is over-expressed.

7. A method of preparing cells for use in the transplantation which are resistant to cyclosporin A or its analogues, comprising the steps of culturing cells to be transplanted in the presence of cyclosporin A or its analogues and recovering viable cells from the cultures.

8. The method as defined in claim 6, wherein the cells are myoblasts.

9. The method as defined in claim 7, wherein the cells are myoblasts.
